(19) United States
(12) Patent Application Publication
(10) Pub. No.: US 2024/0164831 A1
(43) Pub. Date: May 23, 2024

(54) COAGULATION INSTRUMENT

(71) Applicant: Erbe Elektromedizin GmbH, Tuebingen (DE)

(72) Inventors: Lorenz Schoenhaar, Neckartenzlingen (DE); Stefan Kaupp, Tuebingen (DE); Felix Bob, Rottenburg (DE)

(21) Appl. No.: 18/513,378

(22) Filed: Nov. 17, 2023

(30) Foreign Application Priority Data
Nov. 21, 2022 (EP) 22208604.3

(51) Int. Cl. A61B 18/14

(52) U.S. Cl. CPC A61B 18/1445; A61B 2018/00589

(57) ABSTRACT

A coagulation instrument including an insulator unit having at least two plastic bodies between which an anchor section of an electrode is arranged. The anchor section has at least one anchor opening, preferably multiple anchor openings serving for form-fit coupling of the electrode to the insulator unit. At least one of the two plastic bodies is provided with an extension that extends into or through the anchor opening. The extension can extend into the other of the two plastic bodies. Alternatively, at least one of the two plastic bodies can comprise one or more extensions that extend at least into an anchor opening or through the anchor opening and potentially into the other of the two plastic bodies.

This application claims priority to European Patent Application No. 22208604.3, filed Nov. 21, 2022, the entirety of which is incorporated by reference herein

The invention refers to an instrument having at least one electrode, which can be brought into contact with biological tissue for supply of current to the biological tissue.

Instruments of the indicated type, particularly coagulation instruments with coagulation electrodes are generally known. EP 2 853 219 A1 discloses such an instrument having a forceps-like tool that comprises two cooperating jaws, which can be moved toward and away from one another. Each of the two jaws comprises a plastic body on which a coagulation electrode is attached. The coagulation electrode comprises an angled rim serving as anchor section and extending into the plastic body. For anchoring in the plastic body, the anchor section comprises openings, which are passed through by the plastic. In doing so, a form-fit anchoring of the electrode on the plastic body is achieved. The plastic body keeps a tissue contact surface uncovered and comprises centrally a knife slit forming a longitudinal groove. It passes through the electrode and extends into the plastic body.

EP 2 719 352 A1 discloses a similar electrode arrangement with an electrode formed from sheet metal that comprises an anchor section at both sides of its tissue contact surface. Therein anchor openings are introduced that extend in form of through-openings through the anchor section or in form of pockets into the anchor section. In addition, this document discloses spacers on the tissue contact surface in form of electrically non-conductive elevations arranged on the tissue contact surface.

US 2021/0205003 A1 discloses an instrument, the jaw of which comprises an interior plastic body, a metal support being U-shaped in cross-section holding the interior plastic body and an outer plastic body, whereby at the top side of this jaw a tissue contact surface is provided.

US 2012/ 0172873 A1 also discloses a jaw having a structurally reinforcing metal inlay and an interior insulator, which defines the knife channel and which is passed over by an electrode and its anchor section. Outwards the branch is surrounded by an additional plastic body, which contacts the anchor section of the electrode as well as the interior plastic body and the reinforcing metal inlay.

Further jaws made of plastic and metal parts are known from DE 10 2016 100 549 A1 as well as EP 2 092 905 B1.

Instruments of the indicated type exist as single-use instruments as well as sterilizable reusable instruments. During normal use as well as during sterilization the instruments are subject to thermal stresses, e.g. resulting from different thermal expansion coefficients of plastic and metal that could result in gap formation. This can result in difficulties when establishing sterility during sterilization as well as in terms of the mechanical load supporting ability. For this reason, it is desirable in single-use instruments as well as in reusable instruments to reduce the problems accompanying thermal stress.

It is the object of the invention to provide an coagulation instrument improved in this regard.

The coagulation instrument according to the invention comprises at least one jaw on which an electrode is attached that comprises a tissue contact surface. Such an instrument can be, for example, a monopolar instrument having one single electrode or also an element being configured in the type of a forceps having two jaws. Each of the jaws can comprise at least one electrode. Other instrument configurations are possible.

The electrode comprises an anchor section that is provided with at least one anchor opening. The anchor opening can be a through-opening or also a blind opening, for example in the form of a flat pocket or the like. The anchor opening may have been produced by punching, combined punching and bending, drilling, laser removal, etching, eroding, water jet cutting or other machining methods. Particularly the opening can have a smooth edge or an edge having one or more tabs extending away therefrom.

According to an embodiment of the invention, the electrode is held on an insulator unit comprising at least two plastic bodies between which the anchor section extends. The two plastic bodies are connected with the electrode and/or with one another in a form-fit manner. For this purpose, each of the plastic bodies comprises at least one, preferably multiple projections that engage the at least one or more anchor openings of the electrode, this means they project into the anchor openings or pass through the anchor openings. Alternatively, only one of the two plastic bodies comprises projections that extend through the anchor openings and thereby into the other plastic body, whereby in turn an interlocking of the two plastic bodies with one another is achieved.

If the two plastic bodies are interlocked with one another by one or more of the indicated manners, a form-fit is provided between the two plastic bodies, which guarantees a good continuous and reliable connection of the two plastic bodies with one another and thus also a reliable anchoring of the electrode on the insulator unit. In doing so, remarkably reliable coagulation instruments can be manufactured. This is independent form whether the plastic bodies are interlocked with one another in the proximity of, nearby or inside the anchor opening.

The anchor section of the electrode encompasses an interior plastic body and is encompassed itself by an outer plastic body. Both plastic bodies are connected with one another and with the anchor section of the electrode in a form-fit manner in one of the above-mentioned ways. The extensions of the plastic body extending through the openings thereby comprise a length L, which can be at least as long as the thickness D of the anchor section. It is, however, also possible to make the length L of the extensions of the plastic bodies shorter than the thickness D of the anchor section, so that the extensions of the two plastic bodies adjoin one another on a contact surface, which is completely located in the anchor opening.

The presented concept allows the configuration of the plastic bodies from different plastic materials, even if these plastic materials do not form a mechanically rigid welded joint with one another. The manufacturing can be carried out by injection molding during which in a respective injection mold one of the plastic bodies is produced by injection of plastified plastic material first. Thereby anchor structures are formed in the first plastic body, e.g. in form of depressions (blind openings) . After solidification, at least in part of the first plastic body, then the other of the two plastic bodies can be produced by injection of plastified plastic material. The plastic then flows into the anchor structures and thereby forms an interlocking with the first plastic body and the electrode. Particularly in the area of the anchor section of the electrode, a rigid reliable connection is produced between the plastic bodies.

Additional details of advantageous embodiments of the invention are derived from the drawing, the associated description or from the claims. In the drawing embodiments of the invention are disclosed.

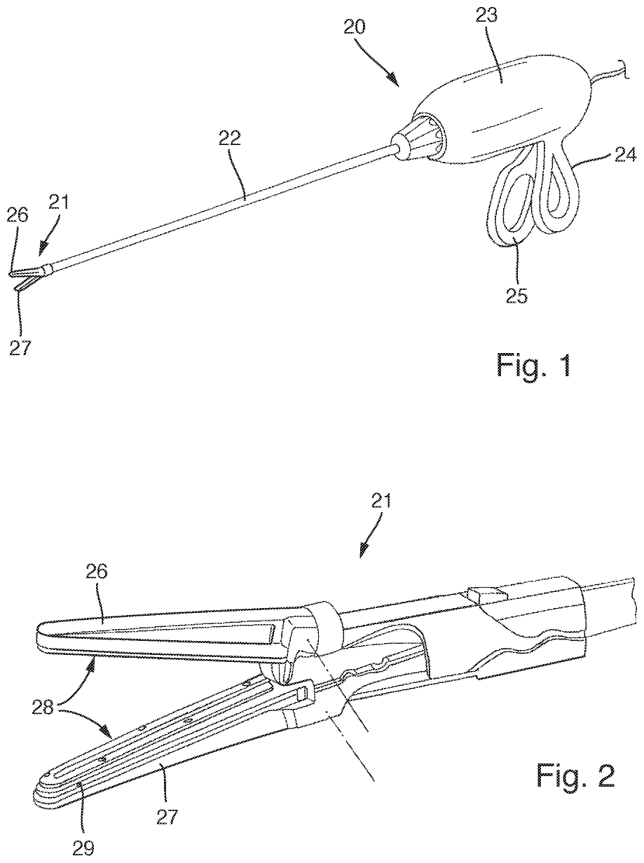

FIG. 1 illustrates a coagulation instrument provided for the laparoscopic use in perspective basic illustration,

FIG. 2 illustrates a tool part of the instrument according to FIG. 1 in perspective illustration,

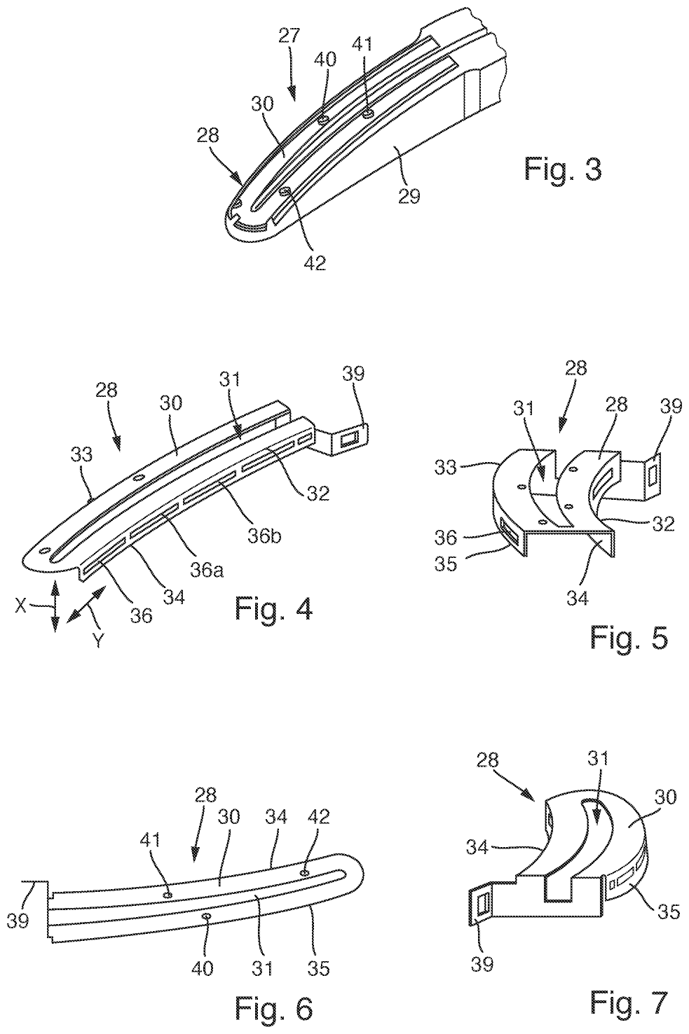

FIG. 3 illustrates a jaw of the tool according to FIG. 2 in perspective illustration,

FIGS. 4 to 7 illustrate different views of an electrode of the tool according to FIGS. 2 and 3,

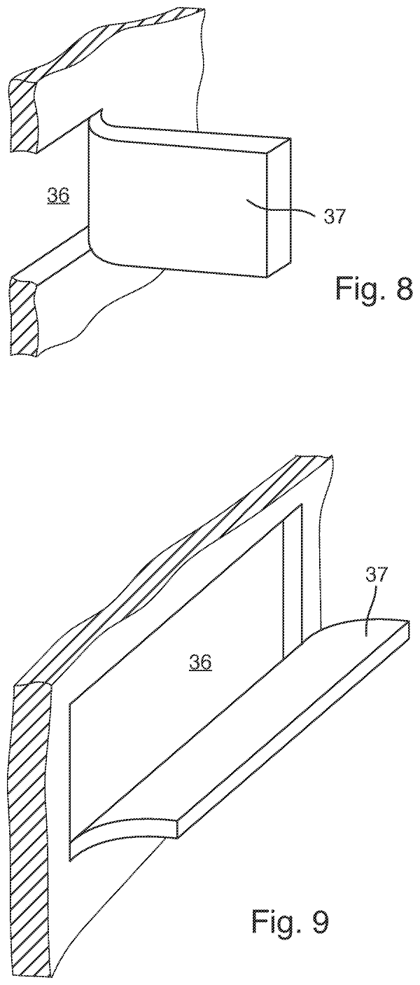

FIG. 8 illustrates an embodiment of an anchor opening of the anchor section of the electrode in perspective illustration in part manufactured by a punching-bending technique,

FIG. 9 illustrates a modified embodiment of an anchor opening of the anchor section of an electrode of an instrument according to the invention manufactured in punching-bending technique,

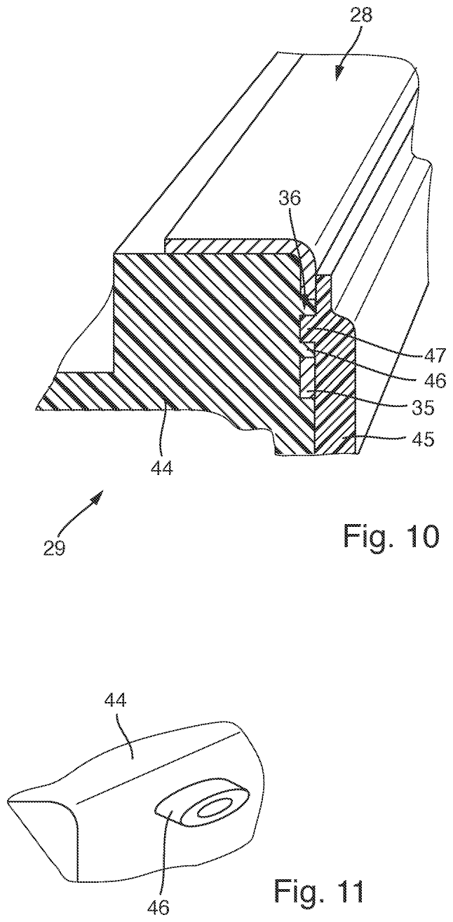

FIG. 10 illustrates the jaw according to FIG. 3 in a cut illustration according to a first embodiment,

FIG. 11 illustrates a sectional illustration of the jaw according to FIG. 10 in a perspective illustration in part,

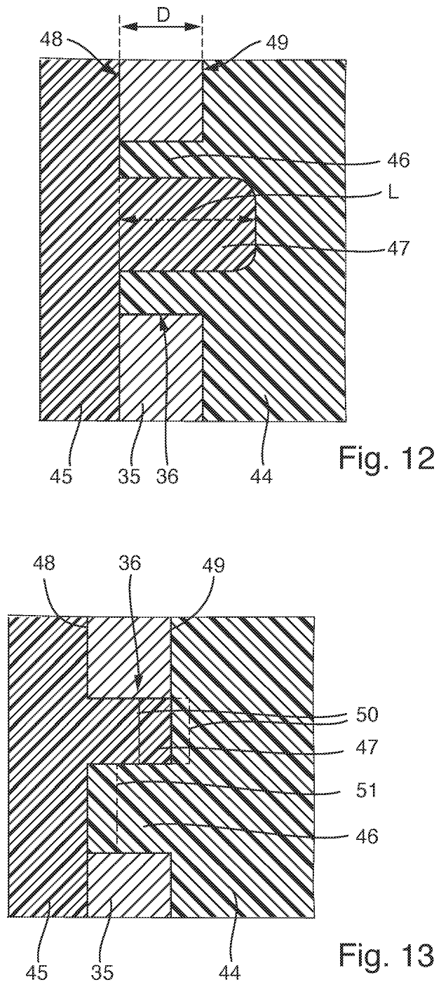

FIG. 12 illustrates a sectional illustration of a jaw in part of an instrument according to the invention according to a second embodiment,

FIG. 13 illustrates the jaw similar to FIG. 10 or 12 according to another embodiment,

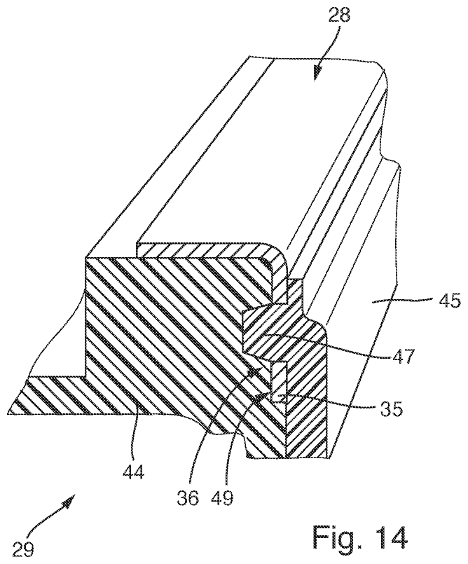

FIG. 14 illustrates the jaw similar to FIG. 12 in a modified embodiment and

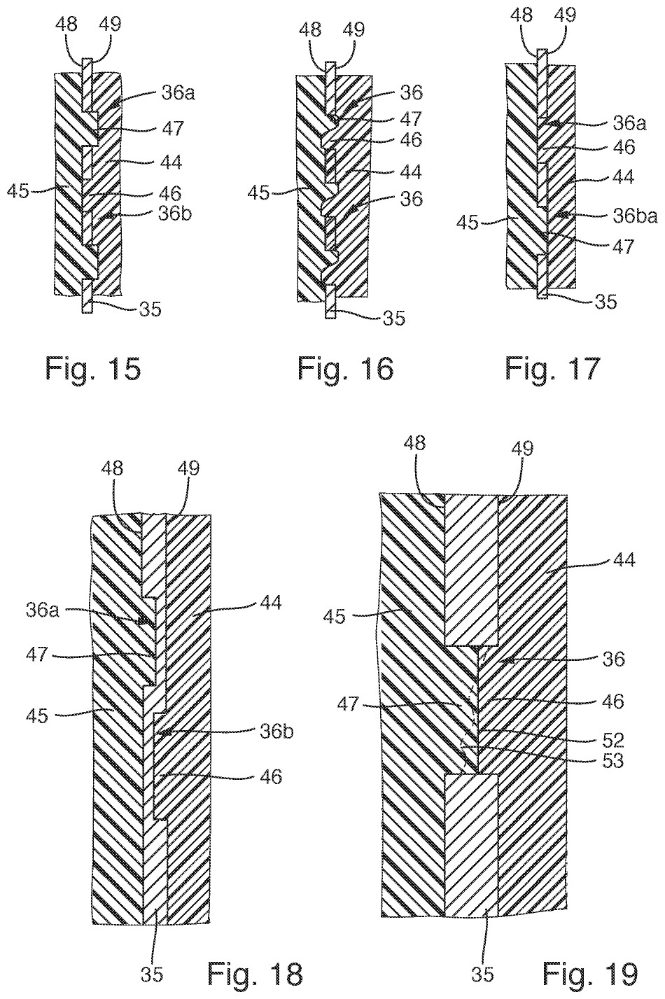

FIGS. 15 to 19 illustrate different embodiments of the jaws based on sectional illustrations of the plastic bodies and the anchor section of the electrode, cut in direction of the anchor openings parallel to the tissue contact surface, in an illustration in part respectively.

A coagulation instrument 20 is illustrated in figure 1 having a forceps-like tool part 21. The tool part 21 is arranged on the distal end of a shank 22, the proximal end of which is supported by a housing 23 having a handle 24 and an operating lever 25. The latter serves for movement of jaws 26, 27 of tool part 21 towards and away from one another in order to thereby grip and coagulate biological tissue, for example particularly hollow vessels, such as blood vessels or the like. For this purpose, both jaws 26, 27 are provided with one electrode 28 respectively on sides facing one another, between which an electrical voltage can be applied so that a current flows through the tissue held therebetween. The tool part 21 can comprise other elements, such as a mechanical movable knife or a cutting electrode.

Instrument 20 illustrated in FIGS. 1 and 2 is a bipolar tool and shall serve here only for sake of illustration. The invention can be used, however, similarly in instruments that are configured for open surgical use or for the endoscopic use. The invention can be used also in instruments, the tool part 21 of which comprises one single electrode only immovably held on the instrument. In addition, the invention can be used in instruments, which are forceps-like configured similar to FIGS. 1 and 2, in which however only one of the electrodes is movably supported.

The jaw 27 is individually illustrated in FIG. 3. It comprises an insulator unit 29 on which the electrode 28 is held.

The electrode 28 is individually illustrated in FIG. 4 as well as in addition in FIGS. 5 to 7. It can be formed from a sheet metal and comprises a tissue contact surface 30. The tissue contact surface 30 can be configured as flat surface, for example. Approximately centered it can be opened by a knife slit 31 extending from the proximal end to the distal end. Such a knife slit 31 can be present, if tool part 21 comprises a mechanical knife that can be forwarded along the knife slit 31 (not illustrated).

At the edge of tissue contact surface 30 at a bending line 32, 33 an anchor section 34, 35 can adjoin, i.e. formed by the two rims of electrode 28 bent downwardly approximately orthogonal, for example. The anchor sections 34, 35 can transition into one another at the distal end of the electrode 28 or, as apparent from FIG. 5, terminate with a distance to one another.

The anchor sections 34, 35 are basically similarly configured, so that the following description of anchor section 35 applies accordingly for the anchor section 34. The anchor section 35 is provided with at least one, preferably multiple anchor openings 36, 36a, 36b that can be configured in a slit-like manner, as illustrated, or alternatively as oval, rounded, circular or longitudinal openings. These openings can be basically configured as through-openings of alternatively as flat pockets.

The anchor openings 36, 36a, 36b can be configured as openings having a smooth edge or also with a tongue 37, 38 appended to the edge, as depicted by FIG. 8 or 9. This tongue 37 or 38 can be produced during creation of the anchor opening 36 by a punching method, in that a separated tongue is bent away from the anchor opening 36. In case of the embodiment according to FIG. 8, this tongue extends orthogonal to the tissue contact surface 30 while in the embodiment according to FIG. 9 it can be orientated substantially parallel thereto. An anchor opening 36, 36a, 36b can also have multiple of such tongues.

Preferably the electrode 28 is configured as sheet metal part and can comprise according to FIGS. 4 to 7 a contact tongue 39 for a connection of an electrical line. Other connection possibilities for connecting of an electrical conductor can be provided as well.

One or more spacers 40, 41, 42 can be provided on the tissue contact surface 30 that are preferably made from an electrically non-conductive material, such as plastic or ceramic. The spacers 40 to 42 can be applied on the tissue contact surface 30. Alternatively, they can have a shank extending through an opening of the tissue contact surface 30.

As shown in FIG. 10, electrode 28 described so far is anchored in the insulator unit 29, which comprises a first, interior plastic body 44 configured in a single or multiple-part manner and an outer single or multi-part plastic body 45, which are connected with the electrode 28 in a form-fit manner.

In the embodiment according to FIG. 10, both plastic bodies 44, 45 are interlocked with one another in the area of the anchor opening 36. For this purpose, the inner plastic body 44 comprises an extension 46 that extends into the anchor opening 36. The second plastic body 45 also comprises an extension 47, also extending into the anchor opening 36. Both extensions 46, 47 can engage one another, as apparent from FIG. 10 in that, for example, first extension 46 comprises a recess in which second extension 47 engages. FIG. 11 individually illustrates plastic body 44 and its hollow pin-like extension 46. It comprises an opening in which the pin-like extension 47 of the other plastic body 45 is formed.

By interlocking of the extensions 46, 47 of the two plastic bodies 44, 45 with one another achieved in this manner and thus concurrently the rigid interlocking with electrode 28 or the anchor section 35, an enduring connection of the two plastic bodies 44, 45 with each other is achieved. This is also the case, if both plastic bodies 44, 45 consist of plastic materials that do not profoundly connect by substance bond, e.g. weld with one another, when they are subsequently input into the injection mold. Thereby the concept according to the invention allows selection of plastic materials for the first and second plastic body 44, 45 without the need to pay much attention to their tendency to connect with one another by substance bond. The selection of plastic materials can in this manner be optimized with regard to biological, mechanical or electrical characteristics, as well as thermal characteristics. For example, plastic body 44 can be made of a wear-resistant fiber-reinforced plastic while plastic body 45 consists of another plastic material that is not fiber-reinforced. While the one plastic material can be selected or optimized with regard to a specific characteristic, e.g. sliding characteristics or stiffness, the other plastic material can be selected or optimized with regard to other characteristics, e.g. sterilization ability or biological compatibility.

In the embodiment according to FIG. 10, extensions 46, 47 are approximately so long that they fill the anchor opening 36, but do not pass therethrough. In modification to this, FIG. 12 shows an embodiment modified in this regard in enlarged illustration. The plastic body 45 abutting against the outer side 48 of anchor section 35 comprises here an extension 47 having a length L that is longer than the thickness D of the anchor section 35 that is to be measured between the outer side 48 and the inner side 49 of the anchor section 35. While extension 47 therefore projects beyond the inner side 49, extension 46 terminates flush with outer side 48. The extensions 46, 47 and thus plastic bodies 44, 45 are interlocked with one another. In addition, they are interlocked with electrode 28 or its anchor section 35. However, it is also possible to define the length L of extension 47 shorter than the thickness D.

In the embodiment according to FIG. 13, plastic bodies 44, 45 comprise again extensions 46, 47 that extend into the anchor opening 36 or therethrough. The extensions 46, 47 can extend adjacent to one another into the anchor opening 36. As shown, the extensions 46, 47 can terminate flush with outer side 48 and inner side 49 or can project beyond it. It is also possible to make the extension 47 slightly shorter, so that it does not reach the inner side 49 coming from the outer side 48, which is indicated in FIG. 13 by dashed lines 50. Additionally or alternatively, extension 46 illustrated flush with outer side 48 in this figure can be made shorter and thus terminate inside anchor opening 36, which is illustrated in FIG. 12 by dashed line 51. Anyway, independent therefrom both plastic bodies 44, 45 are interlocked with one another in the according to FIG. 12. In addition, they are concurrently interlocked with anchor section 35 of electrode 28.

A modified embodiment is illustrated by FIG. 14. There, extension 47 of plastic body 45 passes through anchor opening 36 and extends into plastic body 44. The extension 47 thereby extends beyond inner side 49 of anchor section 35 into plastic body 44, whereby the plastic bodies 44, 45 are interlocked with one another. While the second plastic body 45 is thereby concurrently interlocked with electrode 28, plastic body 44 is not directly interlocked with electrode 28. However, due to the form-fit connection of both plastic bodies 44, 45 with one another, a rigid connection between electrode 28 and the insulator unit 29 is still provided.

The interlocking of the two plastic bodies 44, 45 with one another does not have to be necessarily provided inside one single anchor opening 36. It is also possible to spread the interlocking among multiple anchor openings 36, 36a, 36b, as illustrated by FIG. 15. The anchor openings 36, 36a, 36b can be arranged in a sequence along anchor section 35, as obvious from FIG. 15. For example, extension 47 can extend through anchor opening 36a inwardly, i.e. beyond the inner side 49, while extension 46 extends from inside to outside through anchor opening 36b beyond outer side 48. It is, however, also possible that the extensions 46, 47 share one opening 36 respectively and pass therethrough adjacent to one another, as shown in FIG. 16. Thereby they can terminate flush with the inner side 49 and the outer side 48 or also individually or both project over these sides 48, 49, as illustrated in FIG. 16.

Following the embodiment according to FIG. 15 it is also possible to arrange the extensions 46, 47 alternatingly in the sequence of anchor openings 36a, 36b, whereby one or both extensions 46, 47 terminate with outer side 48 and inner side 49 respectively. In this embodiment both plastic bodies 44, 45 are interlocked with anchor section 35 of electrode 28, but not with one another. On the contrary, in the embodiments according to FIGS. 15 and 16, plastic bodies 44, 45 are interlocked with one another and among each other as well as with the anchor section.

FIG. 18 and FIG. 19 illustrate additional embodiments in which the plastic bodies 44, 45 are interlocked with anchor section 35 and thus the electrode 28, however not among one another. In the embodiment according to FIG. 18, anchor openings 36a, 36b are configured as flat pockets into which extensions 46, 47 extend. Thereby an interlocking of plastic body 44 with anchor section 35 and thus electrode 28 is achieved. Similarly, an interlocking of plastic body 45 with anchor section 35 and thus electrode 28 is achieved.

In the embodiment according to FIG. 19, interlocking of extensions 46, 47 with anchor section 35 is achieved in that both extensions 46, 47 extend into the anchor opening 36 and get into contact therein. Thereby they can get into contact at a surface 52 located inside anchor opening 36. The surface 52 can be a planar surface or also a non-planar, e.g. curved contact surface. It can be parallel to the inner side 49 and the outer side 48 or also orientated in an angle thereto and thereby extend obliquely through the anchor opening 36. Such a non-planar and obliquely positioned contact line is indicated in FIG. 19 in form of a dashed line 53.

A coagulation instrument according to the invention comprises an insulator unit 29 consisting of at least two plastic bodies 44, 45 between which an anchor section 35 of an electrode 28 is arranged. The anchor section 35 comprises at least one anchor opening, preferably multiple anchor openings serving for form-fit coupling of the electrode to the insulator unit 29. For this purpose, at least one of the plastic bodies 44, 45 is provided with an extension 47 that extends into or through the anchor opening 36. It can extend into the other plastic body 44. Alternatively, the plastic body 44 can comprise one or more extensions 46 that extend at least into an anchor opening 36 or through the anchor opening and potentially into the other plastic body 45.

1. A coagulation instrument for coagulation and/or fusion of biological tissue comprising:
at least one jaw comprising an electrode having a tissue contact surface and at least one anchor section, the anchor section having at least one anchor opening; and
an insulator unit comprising two plastic bodies between which the anchor section extends, wherein both plastic bodies are connected in form-fit manner with the electrode or with one another.

2. A coagulation instrument according to claim 1, wherein the plastic bodies are interlocked with one another.

3. A coagulation instrument according to claim 1, wherein the plastic bodies are interlocked with one another near the anchor opening.

4. A coagulation instrument according to claim 1, wherein the plastic bodies are interlocked with one another inside the anchor opening.

5. A coagulation instrument according to claim 1, wherein the anchor section of the electrode embraces one of the plastic bodies.

6. A coagulation instrument according to claim 5, wherein the plastic body embraced by the anchor section comprises extensions extending into or through openings of the anchor section.

7. A coagulation instrument according to claim 1, wherein one of the plastic bodies embraces the anchor section of the electrode.

8. A coagulation instrument according to claim 7, wherein the plastic body embracing the anchor section comprises extensions extending into or through openings of the anchor section.

9. A coagulation instrument according to claim 8, wherein the extensions have a length (L), which is at least as long as the thickness (D) of the anchor section.

10. A coagulation instrument according to claim 1, wherein the anchor section comprises an angled rim projecting from the tissue contact surface, wherein the rim is in contact with one of the plastic bodies with one side and with the other of the plastic bodies with its opposite side.

11. A coagulation instrument according to claim 1, wherein the anchor opening is a through-opening.

12. A coagulation instrument according to claim 1, wherein one of the plastic bodies is a fiber-reinforced plastic body.

13. A coagulation instrument according to claim 1, wherein one of the plastic bodies surrounds a knife channel.

14. A coagulation instrument according to claim 1, wherein the plastic bodies are made of different plastic materials.

15. A coagulation instrument according to claim 1, wherein the plastic bodies are connected to one another in substance bond manner.
